(19) United States
(12) Patent Application Publication
(10) Pub. No.: US 2008/0292695 A1
(43) Pub. Date: Nov. 27, 2008

(54) CARVEDILOL FORMS, COMPOSITIONS, AND METHODS OF PREPARATION THEREOF

(76) Inventors: Kristin Arnold, Morrisville, PA (US); E. Brendan Magrab, Westfield, NJ (US); David F. Erkoboni, Pennington, NJ (US); Zhongshui Yu, Bridgewater, NJ (US); Siva Rama K. Nutalapati, Princeton, NJ (US); Rakeshkumar K. Lad, Marlton, NJ (US)

Correspondence Address: CANTOR COLBURN, LLP, 20 Church Street, 22nd Floor, Hartford, CT, 06103, US

(21) Appl. No.: 11/949,158

(22) Filed: Dec. 3, 2007

Related U.S. Application Data
(60) Provisional application No. 60/872,097, filed on Dec. 1, 2006.

(51) Int. Cl. A61K 31/403; A61K 9/52; A61K 9/28; A61P 9/00

(52) U.S. Cl. 424/457; 514/411; 424/468

(57) ABSTRACT

Disclosed are amorphous carvedilol salt forms, controlled-release carvedilol compositions, and methods of preparing the forms and compositions.

### CROSS REFERENCE TO RELATED APPLICATION

This application claims priority from U.S. Provisional Application Ser. No. 60/872,097 filed Dec. 1, 2006, which is hereby incorporated by reference in its entirety.

### BACKGROUND

Carvedilol, (±)-1-(carbazol-4-yloxy)-3-[[2-(o-methoxyphenoxy)ethyl]amino]-2-propanol, is a known β-blocker used in the treatment of angina, hypertension, and congestive heart failure.

Carvedilol has limited aqueous solubility, especially in basic aqueous media, which limits the active agent's absorption in the gastrointestinal (GI) tract of the patient. Prior attempts to improve the solubility of carvedilol include the use of a solubilizer with the active agent, or the formation of various carvedilol salt forms which have varying degrees of solubility when compared to carvedilol free base. Still, there remains a need in the art for carvedilol compositions having improved aqueous solubility.

Controlled-release compositions that can provide improved release profiles unattainable with immediate-release dosage forms are also needed. Such controlled-release compositions could provide a once daily dosing of a particular active agent thereby increasing patient compliance and the probability of a successful treatment for the targeted disease, while at the same time decreasing the likelihood of missed doses.

Therefore, there is a continuing need for improved controlled-release compositions containing carvedilol.

### SUMMARY

In one embodiment, a controlled-release composition comprises carvedilol and a release-retarding excipient, wherein the controlled-release composition exhibits a single phase release (single Tₘₐₓ) within the first 10 hours after oral administration to a patient.

In another embodiment, a controlled-release composition comprises carvedilol and a release-retarding excipient, wherein the controlled-release composition exhibits a first peak plasma Tₘₐₓ₁ of greater than 4 hours, and a second peak plasma Tₘₐₓ₂ of greater than 10 hours.

In yet another embodiment, an amorphous carvedilol phosphate complex comprises amorphous carvedilol phosphate salt and a complexing agent.

These and other embodiments, advantages and features of the present invention become clear when detailed description and examples are provided in subsequent sections.

### DETAILED DESCRIPTION

Discloses herein are amorphous carvedilol salt forms, controlled-release carvedilol compositions, and methods of preparing the forms and compositions.

Generally, the controlled-release carvedilol composition comprises carvedilol and a release-retarding material in the form of a matrix or coating. The controlled-release composition can be prepared as a monolithic form, a layered form comprising two or more layers, a coated form, a plurality of subunits; specifically as a plurality of subunits. The controlled-release subunits can further be combined in a single dosage form with an optional immediate-release portion in the form of immediate-release subunits (e.g., in a capsule or compressed into a tablet using compressible binders), immediate-release powders of the active agent, or immediate-release coatings of the active agent substantially surrounding the controlled-release subunits.

In one embodiment, two or more types of controlled-release subunits can be combined in one composition, where each subunit provides a different release profile (e.g., a combination of extended-release and delayed-release subunits in a single composition, optionally in combination with an immediate-release portion as previously discussed).

The controlled-release composition can be used to treat a patient for hypertension, congestive heart failure, or angina by administering an effective amount of carvedilol.

An “active agent” means a compound, element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates) of the free compound or salt, crystalline forms, non-crystalline forms (amorphous), and any polymorphs of the compound are contemplated herein. Compounds may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In addition, compounds with carbon-carbon double bonds may occur in Z- and E-forms, with all isomeric forms of the compounds. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

“Pharmaceutically acceptable salts” include derivatives of carvedilol, wherein carvedilol is modified by making acid addition salts thereof, and further refers to pharmaceutically acceptable solvates, including hydrates, crystalline forms, non-crystalline forms, polymorphs, and stereoisomers of such salts. Examples of pharmaceutically acceptable salts include, but are not limited to, mineral or organic acid addition salts, for example, those derived from inorganic acids such as hydrochloric, hydrobromic, sulfuric, sulfamic, phosphoric, nitric and the like. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like.

Exemplary carvedilol salts include carvedilol benzoate, carvedilol citrate, carvedilol glutarate, carvedilol hydrobromide, carvedilol hydrochloride, carvedilol hydrogen phosphate, carvedilol dihydrogen phosphate, carvedilol lactate, carvedilol mandelate, carvedilol maleate, carvedilol mesylate, carvedilol oxalate, carvedilol sulfate, or a hydrate or solvate of the foregoing salts and including all crystalline and non-crystalline forms thereof. Suitable carvedilol salts can be found in U.S. Patent Application Publication Nos. 2005/0277689 and 2005/240027, each of which is incorporated by reference herein.

Carvedilol and its salts may exist in one or more crystalline forms. Known polymorphs of carvedilol include those disclosed in Patent Application Publication Nos. 2004/0225132, 2004/152756, 2003/119893, and 2004/198812, as well as U.S. Pat. Nos. 6,730,326 and 4,503,067, each of which is incorporated by reference herein.

“Carvedilol” as used herein is inclusive of all pharmaceutically acceptable salt forms, crystalline forms, amorphous form, polymorphic forms, solvates, and hydrates unless specifically indicated otherwise.

A “dosage form” means a unit of administration of an active agent. Examples of dosage forms include tablets, capsules, injections, suspensions, liquids, emulsions, creams, ointments, suppositories, inhalable forms, transdermal forms, and the like.

By “oral dosage form” is meant to include a unit dosage form for oral administration. An oral dosage form may optionally comprise a plurality of subunits such as, for example, microcapsules or microtablets. Multiple subunits may be packaged for administration in a single dose. Other dosage forms for oral administration include, for example, suspension, an emulsion, orally disintegrating tablets including effervescent tablets, chewable tablets, gastro-resistant tablets, soft capsules, hard capsules, gastro-resistant capsules, coated granules, gastro-resistant granules, modified-release granules, osmotic pumps, and the like.

By “subunit” is meant to include a composition, mixture, particle, pellet, microcapsules or microtablets, etc., that can provide an oral dosage form alone or when combined with other subunits.

By “immediate-release” is meant a conventional or non-modified release in which greater then or equal to about 75% of the active agent is released within two hours of administration, specifically within one hour of administration.

By “controlled-release” is meant a dosage form in which the release of the active agent is controlled or modified over a period of time. Controlled can mean, for example, sustained-, delayed- or pulsed-release at a particular time. Alternatively, controlled can mean that the release of the active agent is extended for longer than it would be in an immediate-release dosage form, e.g., at least over several hours.

Dosage forms can be combination dosage forms having both immediate-release and controlled-release characteristics, for example, a combination of immediate-release subunits and controlled-release subunits. The immediate-release portion of a combination dosage form may be provided as a loading dose.

Disclosed herein are controlled-release carvedilol oral dosage compositions, particularly solid oral dosage compositions. The solid, oral controlled-release carvedilol dosage compositions generally comprise carvedilol and a release-retarding material wherein the release-retarding material is in the form of a matrix or a coating. The combination of carvedilol and release-retarding material can be in the form of a monolithic tablet, a layered tablet; or subunit form such as a granule, a microtablet, a minitablet, a caplet, a pellet (as used herein “pellet” means a spherical granule prepared by extrusion and spheronization, and is equivalent to bead, spheroid, and microsphere), a particle, an active agent core, or other multiparticulate system prepared with a release-retarding matrix material or a release-retarding coating material. Examples of extended-release compositions which are suitable for use with carvedilol include those provided in Sustained Release Medications, Chemical Technology Review No. 177. Ed. J. C. Johnson. Noyes Data Corporation 1980; and Controlled Drug Delivery, Fundamentals and Applications, 2nd Edition. Eds. J. R. Robinson, V. H. L. Lee. Mercel Deklcer Inc. New York 1987. Additional forms are described in U.S. Pat. Nos. 5,102,666 and 5,422,123.

The release-retarding material of the matrix or coating can be selectively chosen so as to achieve, in combination with the other stated properties, a desired in vitro or in vivo release profile.

Exemplary release-retarding matrix materials, specifically to prepare the subunits, include for example an acrylic polymer, an alkylcellulose, shellac, zein, hydrogenated vegetable oil, hydrogenated castor oil, polyvinylpyrrolidine, a vinyl acetate copolymer, polyethylene oxide, or a combination comprising at least one of the foregoing materials. The extended-release oral composition can contain about 1.0 wt % to about 80 wt % of the release-retarding matrix material based on the total weight of the oral composition, specifically about 10 wt % to about 70 wt %, and more specifically about 20 wt % to about 50 wt %.

Suitable acrylic polymers that can be used as release-retarding matrix materials include, for example, an acrylic acid and methacrylic acid copolymer, a methyl methacrylate copolymer, an ethoxyethyl methacrylate polymer, a cyanoethyl methacrylate polymer, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, or a combination comprising at least one of the foregoing materials. The acrylic polymer may comprise methacrylate copolymers described in NF XXIV as fully polymerized copolymers of acrylic and methacrylic acid esters with a low content of quaternary ammonium groups. Exemplary copolymers of acrylic and methacrylic acid esters with a low content of quaternary ammonium groups include EUDRAGIT RS, and EUDRAGIT RL commercially available from Rohm Pharma GmbH, Germany. Other suitable copolymers of acrylic and methacrylic acid esters include EUDRAGIT NE30D, EUDRAGIT L, EUDRAGIT S (e.g., EUDRAGIT S 100), and the like, all of which are commercially available from Rohm Pharma GmbH, Germany.

Suitable alkylcelluloses include, for example, methylcellulose, ethylcellulose, and the like. Those skilled in the art will appreciate that other cellulosic polymers, including other alkyl cellulosic polymers, can be substituted for part or all of the ethylcellulose. Other release-retarding matrix materials include modified celluloses such as a carboxymethylcellulose, a low molecular weight hydroxypropylmethylcellulose, a medium viscosity hydroxypropylmethylcellulose, a crosslinked sodium carboxymethylcellulose, a crosslinked hydroxypropylcellulose, a high molecular weight hydroxypropylmethylcellulose, or a combination comprising at least one of the foregoing materials.

Other suitable release-retarding matrix materials include a neutral or synthetic wax, a fatty alcohol (such as lauryl, myristyl, stearyl, cetyl or specifically cetostearyl alcohol), a fatty acid, including fatty acid esters, fatty acid glycerides (mono-, di-, and tri-glycerides), a hydrogenated fat, a hydrocarbon wax, a normal wax, stearic acid, stearyl alcohol, hydrophobic and hydrophilic materials having hydrocarbon backbones, or a combination comprising at least one of the foregoing materials. Suitable waxes include beeswax, glycowax, castor wax, carnauba wax and wax-like substances, e.g., material normally solid at room temperature and having a melting point of from about 30° C. to about 100° C., or a combination comprising at least one of the foregoing waxes.

Other suitable release-retarding matrix material can include a digestible, long chain (e.g., C₈-C₅₀, specifically C₁₂-C₄₀) substituted or unsubstituted hydrocarbon, such as fatty acids, fatty alcohols, glyceryl esters of fatty acids, mineral and vegetable oils, waxes, or a combination comprising at least one of the foregoing materials. Hydrocarbons having a melting point of between about 25° C. and about 90° C. may be used. Specifically, long chain hydrocarbon materials, fatty (aliphatic) alcohols can be used.

Still further suitable release-retarding matrix materials include a polylactic acid, a polyglycolic acid, a co-polymer of lactic and glycolic acid, a carboxymethyl starch, a potassium methacrylate/divinylbenzene copolymer, a crosslinked polyvinylpyrrolidone, a high molecular weight polyvinylalcohol, a low molecular weight polyvinylalcohol, a polyethylene glycol, a non-crosslinked polyvinylpyrrolidone, a medium viscosity polyvinylalcohol, or a combination comprising at least one of the foregoing materials.

Release-modifying agents, which can be used to adjust the release properties of the release-retarding material, can optionally be used. The release-modifying agent can, for example, function as a pore-former. The pore former can be organic or inorganic, and include materials that can be dissolved, extracted or leached from the material in the environment of use. The pore-former can comprise one or more hydrophilic polymers, such as hydroxypropylmethylcellulose, hydroxypropylcellulose, polycarbonates comprised of linear polyesters of carbonic acid in which carbonate groups reoccur in the polymer chain, or a combination comprising at least one of the foregoing release-modifying agents. Alternatively, the pore-former may be a small molecule such as a mono- or disaccharide (e.g., lactose), a sugar alcohol, or a metal stearate, or a combination comprising at least one of the foregoing release-modifying agents.

The release-retarding matrix material can also optionally be combined with other additives such as an erosion-promoting agent (e.g., starch and gums); or a semi-permeable polymer. In addition to the above ingredients, the controlled-release composition prepared from a controlled-release matrix may also contain suitable quantities of other materials, e.g., diluents, lubricants, binders, granulating aids, colorants, flavorants and glidants that are conventional in the pharmaceutical art. The release-retarding material can also include an exit means comprising a passageway, orifice, or the like. The passageway can have any shape, such as round, triangular, square, elliptical, irregular, etc.

The controlled-release composition comprising carvedilol and a release-retarding matrix material may be prepared by, for example, dry granulation or wet granulation followed by compression or compaction, melt extrusion and spheronization, layering (e.g., spray layering suspension or solution), and the like. Examples of such techniques include direct compression, using appropriate punches and dies, the punches and dies are fitted to a suitable rotary tableting press; injection or compression molding using suitable molds fitted to a compression unit, granulation followed by compression; and extrusion in the form of a paste, into a mold or to an extrudate to be cut into lengths.

The subunits can be prepared by compression into a compressed form (e.g., small tablets) using conventional tableting equipment using standard techniques. Techniques and compositions for making tablets (compressed and molded) are described in Remington's Pharmaceutical Sciences, (Aurther Osol., editor), 1553-1593 (1980).

Layering techniques suitable to prepare the subunits comprising the controlled-release matrix material include coating inert cores with a layering solution or dispersion of carvedilol and the release-retarding matrix material. Repeated layering can be used to build the subunit size and increase active agent amount.

Exemplary liquids that can be used to prepare the layering dispersion or solution include water, lower alkyl alcohols (e.g., methanol, ethanol, n-propanol, isopropanol, etc.), lower alkyl ketones or acetates (e.g., acetone, ethyl acetate, etc.), lower allyl ethers (e.g., ethyl ether, tetrahydrofuran, etc.), acetonitrile, lower halogenated alkyls (e.g., dichloromethane, etc.), or a combination comprising at least one of the foregoing solvents.

Materials suitable for use as the inert cores include pharmaceutically acceptable materials that have appropriate dimensions and firmness. Examples of such materials are polymers e.g. plastic resins; inorganic substances, e.g. silica, glass, hydroxyapatite, salts (sodium or potassium chloride, calcium or magnesium carbonate) and the like; organic substances, e.g. activated carbon, acids (citric, fumaric, tartaric, ascorbic and the like acids), and saccharides and derivatives thereof. The saccharides include sugars, oligosaccharides, polysaccharides and their derivatives, for example, glucose, rhamnose, galactose, lactose, sucrose, mannitol, sorbitol, dextrin, maltodextrin, cellulose, microcrystalline cellulose, sodium carboxymethyl cellulose, starches (maize, rice, potato, wheat, tapioca) and the like.

The inert core can have a diameter of about 250 to about 2000 micrometers, specifically about 600 to about 1500 micrometers, and yet more specifically about 750 to about 1000 micrometers.

The carvedilol can be processed with a release-retarding matrix material and formed into a plurality of subunits. A variety of subunits can be prepared, each exhibiting different characteristics, such as pH dependence of release, time for release in various media (e.g., acid, base, simulated intestinal fluid), release in vivo, size, and composition. The different subunits can then be combined to result in a composition formulated to meet a targeted release profile in vivo or in vitro. Immediate-release subunits can also be combined with controlled-release subunits into a single composition.

The subunits can be presented in a capsule, blended with a compressible binder and compressed into tablets, or prepared into other suitable unit dosage forms.

In one embodiment, the controlled-release composition comprises a plurality of coated subunits, wherein the coating comprises release-retarding coating material. The subunit can be an immediate-release subunit or can comprise a release-retarding matrix material within the subunit itself, as described above. Still further, the coated subunits can be blended with a release-retarding matrix material and prepared into a composition via compression or a similar process.

In addition, the extended-release profile of carvedilol (either in vivo or in vitro) can be altered, for example, by using more than one release-retarding coating material, varying the thickness of the release-retarding coating material, changing the particular release-retarding coating material used, altering the relative amounts of release-retarding coating material, altering the manner in which the plasticizer is added (e.g., when the extended-release coating is derived from an aqueous dispersion of hydrophobic polymer), by varying the amount of plasticizer relative to release-retarding coating material, by the inclusion of additional ingredients or excipients, by altering the method of manufacture, etc.

Exemplary release-retarding coating materials include a water insoluble alkyl cellulose, (e.g. ethylcellulose, such as AQUACOAT, a 30% dispersion available from FMC, Philadelphia, Pa.; or SURELEASE a 25% dispersion available from Colorcon, West Point, Pa.); water insoluble material such as a wax, either alone or in admixture with a fatty alcohol, or shellac or zein; polyvinyl acetate phthalate (PVAP); hydroxypropylmethyl-cellulose acetate succinate (HPMCAS); cellulose acetate phthalate (CAP); methacrylic acid copolymer; hydroxypropyl methylcellulose succinate; cellulose acetate succinate; cellulose acetate hexahydrophthalate; hydroxypropyl methylcellulose hexahydrophthalate; hydroxypropyl methylcellulose phthalate (HPMCP); cellulose propionate phthalate; cellulose acetate maleate; cellulose acetate trimellitate; cellulose acetate butyrate; cellulose acetate propionate; copolymers of acrylic and methacrylic acid as disclosed above, (e.g., poly(methacrylic acid, methyl methacrylate) 1:1, acid number 300 to 330 and also known as EUDRAGIT L, which is an anionic copolymer based on methacrylate and available as a powder also known as methacrylic acid copolymer, type A NF), methacrylic acid-methyl methacrylate copolymer 1:2 (e.g. EUDRAGIT S), ethyl methacrylate-methylmethacrylate-chlorotrimethylammonium ethyl methacrylate copolymer, methacrylic acid-ethyl acrylate copolymer 1:1 (e.g. EUDRAGIT L 30 D-55), and the like, or a combination comprising at least one of the foregoing materials.

A copolymer of acrylic and methacrylic acid ester (methacrylic acid: acrylic acid ethyl ester 1:1 copolymer solid substance of the acrylic dispersion sold under the trade designation EUDRAGIT L-100-55) may be suitable.

Optionally the release-retarding coating may further comprise a water-soluble component such as an agent that can form channels through the coating upon the hydration or dissolution of the water-soluble component. Specifically, the water-soluble component can be a hydroxyalkylcellulose, hydroxyalkyl(alkylcellulose), carboxymethylcellulose, salts thereof, or a combination comprising at least one of the foregoing. Particular examples of these water-soluble components include hydroxyethylcellulose, hydroxypropylcellulose, hydroxyethylmethylcellulose, hydroxypropylmethylcellulose, carboxymethylcellulose, sodium carboxymethylcellulose, or a combination comprising at least one of the foregoing materials.

The total of the water-soluble component and release-retarding coating material in the controlled-release coating can be in weight ratios of about 1:4 to about 2:1, specifically about 1:2 to about 1:1, and more specifically in a ratio of about 2:3. Other ratios can be used to modify the speed with which the coating permits release of the active agent.

The inclusion of an effective amount of a plasticizer in the controlled-release coating can improve the physical properties of the coating. For example, because ethyl cellulose has a relatively high glass transition temperature and does not form flexible films under normal coating conditions, it may be advantageous to add plasticizer to the ethyl cellulose before using the same as a coating material. Generally, the amount of plasticizer included in a coating solution is based on the concentration of the polymer, e.g., most often from about 1 wt % to about 50 wt % of the polymer. Concentrations of the plasticizer, however, can be determined by routine experimentation.

Examples of plasticizers for ethyl cellulose and other celluloses include dibutyl sebacate, diethyl phthalate, triethyl citrate, tributyl citrate, triacetin, or a combination comprising at least one of the foregoing plasticizers; although it is possible that other water-insoluble plasticizers (such as acetylated monoglycerides, phthalate esters, castor oil, etc.) can be used.

Examples of plasticizers for acrylic polymers include citric acid esters such as triethyl citrate NF, tributyl citrate, dibutyl phthalate, 1,2-propylene glycol, polyethylene glycols, propylene glycol, diethyl phthalate, castor oil, triacetin, or a combination comprising at least one of the foregoing plasticizers; although it is possible that other plasticizers (such as acetylated monoglycerides, phthalate esters, castor oil, etc.) can be used.

Suitable methods can be used to apply the controlled-release coating material to the surface of the subunits. Processes such as simple or complex coacervation, interfacial polymerization, liquid drying, thermal and ionic gelation, spray drying, spray chilling, fluidized bed coating, pan coating, or electrostatic deposition may be used.

To obtain controlled-release of the active agent from the subunit in a manner sufficient to provide a therapeutic effect for sustained durations, the subunit can be coated with an amount of release-retarding coating material sufficient to obtain a weight gain level from about 2 wt % to about 30 wt %, specifically about 3 wt % to about 28 wt %, more specifically about 4 wt % to about 25 wt %, yet more specifically about 6 to about 20 wt %, and still yet more specifically about 12 to about 16 wt %, although the coat can be greater or lesser depending upon the physical properties of the active agent utilized and the desired release rate, among other things. Moreover, there can be more than one release-retarding material used in the coat, as well as various other pharmaceutical excipients.

In one embodiment, the subunit is coated with about 2 to about 25 wt % ethyl cellulose, specifically about 5 to about 22 wt %, more specifically about 10 to about 18 wt %, and still yet more specifically about 12 to about 16 wt % of ethyl cellulose (e.g. Surlease) based on the total weight of the subunit.

In another embodiment, the subunit is coated with about 1 to about 20 wt % copolymers of acrylic and methacrylic acid esters (e.g., EUDRAGIT L30 copolymer of acrylic and methacrylic acid ester, L100, S100, or combinations), specifically about 2 to about 16 wt %, more specifically about 3 to about 12 wt %, and still yet more specifically about 5 to about 10 wt % of copolymer of acrylic and methacrylic acid ester based on the total weight of the subunit.

Exemplary controlled-release coated subunits can be prepared by coating an inert core with a combination of carvedilol and a water soluble film forming polymer to form a carvedilol coated core, optionally coating the carvedilol coated core with a barrier layer, and finally top-coating with a release-retarding coating material.

Materials suitable for use as the inert cores include pharmaceutically acceptable materials that have appropriate dimensions and firmness. Examples of such materials are polymers e.g. plastic resins; inorganic substances, e.g. silica, glass, hydroxyapatite, salts (sodium or potassium chloride, calcium or magnesium carbonate) and the like; organic substances, e.g. activated carbon, acids (citric, fumaric, tartaric, ascorbic and the like acids), and saccharides and derivatives thereof. Particularly suitable materials are saccharides such as sugars, oligosaccharides, polysaccharides and their derivatives, for example, glucose, rhanmose, galactose, lactose, sucrose, mannitol, sorbitol, dextrin, maltodextrin, cellulose, microcrystalline cellulose, sodium carboxymethyl cellulose, starches (maize, rice, potato, wheat, tapioca) and the like saccharides.

The inert core can have a diameter of about 250 to about 2000 micrometers, specifically about 600 to about 1500 micrometers, and yet more specifically about 750 to about 1000 micrometers.

Still other exemplary coated subunits can be prepared without an inert core, but rather formed from granules, compressed tablets of varying size, or pellets of carvedilol and a pharmaceutically acceptable excipient (e.g., microcrystalline cellulose, and the like). Such granules, compressed tablets, and pellets can be prepared according to art-known methods of granulation, compression, and extrusion/spheronization.

In one embodiment, the controlled-release subunit does not contain an orifice created in the controlled-release coating. Rather, the controlled-release coating substantially surrounds the subunit, thereby having no area free of the controlled-release coating.

The controlled-release subunits can be prepared to have any desired size depending upon the choice of release profile, final dosage form, and other considerations. In one embodiment, the controlled-release subunits have a mean diameter of greater than about 2100 micrometers, specifically greater than about 3000 micrometers; and more specifically greater than about 5000 micrometers as measured by the longest dimension. In another embodiment, the controlled-release subunits have a mean diameter of about 700 to about 7500 micrometers, specifically about 1000 to about 5000 micrometers, and yet more specifically about 2500 to about 5000 micrometers as measured by the longest dimension.

In order to achieve the desired pharmacokinetic profile, the compositions may comprise subunits that release carvedilol at different rates, a kind that releases carvedilol slowly, and a kind that releases carvedilol more rapidly, in particular one kind that releases the active ingredient immediately, e.g. subunits as described that lack the release-retarding material.

In one embodiment, part of the total amount of carvedilol in the composition is present in an immediate-release portion. Any type of immediate-release compositions are contemplated, for example, as subunits lacking a release-retarding material coating/matrix, immediate-release carvedilol powder or particles which can be blended with controlled-release subunits, or as a topcoat coating the controlled-release subunits or coating the entire controlled-release composition.

The subunits can be filled in capsules such that a therapeutically effective amount of the active ingredient is available per dosage form. In one embodiment, the subunit-filled capsules provide a pharmacokinetic profile wherein a first peak plasma Tₘₐₓ₁ of greater than 4 hours, and a second peak plasma Tₘₐₓ₂ of greater than 10 hours is obtained.

The different subunits may be filled consecutively in the capsules, or they may be premixed and the thus obtained premix may be filled into the capsules (taking into account possible segregation).

Alternatively, the controlled-release subunits may further comprise a top-coat of a water-soluble polymer as described hereinbefore and carvedilol which is released practically immediately upon ingestion and thus ensures a rapid onset of action.

The composition can be prepared by various conventional mixing, comminution and fabrication techniques readily apparent to those skilled in the art of pharmaceutical formulations. Examples of such techniques include direct compression, using appropriate punches and dies, the punches and dies are fitted to a suitable rotary tableting press; injection or compression molding using suitable molds fitted to a compression unit, granulation followed by compression; and extrusion in the form of a paste, into a mold or to an extrudate to be cut into lengths.

The subunits can be compressed into an oral tablet using conventional tableting equipment using standard techniques. Techniques and compositions for making tablets (compressed and molded) are described in Remington's Pharmaceutical Sciences, (Aurther Osol., editor), 1553-1593 (1980).

The composition may be in the form of subunits enclosed inside a capsule, e.g. a gelatin capsule. For this, a gelatin capsule employed in the pharmaceutical formulation field can be used, such as the hard gelatin capsule known as CAPSUGEL, available from Pfizer.

The controlled-release carvedilol compositions can be prepared into a variety of dosage forms besides a plurality of subunits. Other controlled-release compositions can include those that are easily administered for those patients that have difficulty with oral solid dosage compositions, such as tablets and capsules. Such compositions would be useful for elderly patients who require dosage forms that are easy to swallow. Easily administered formulations, such as chewable tablets, gummy forms, candy forms, sprinkle forms, liquid or semi-solid formulations (e.g. suspensions or emulsions), taste-masked formulations, and fast dissolve tablets, are thus desirable.

For easy administration, the controlled-release composition can be a chewable tablet containing the active agent. A chewable tablet generally comprises a chewable base and optionally a sweetener. The chewable base comprises an excipient such as, for example, mannitol, sorbitol, lactose, or a combination comprising at least one of the foregoing excipients. The optional sweetener used in the chewable composition may be, for example, digestible sugars, sucrose, liquid glucose, sorbitol, dextrose, isomalt, liquid maltitol, aspartame, lactose, or a combination comprising at least one of the foregoing sweeteners. In certain cases, the chewable base and the sweetener may be the same component. The chewable base and optional sweetener may comprise about 50 wt % to about 90 wt % of the total weight of the composition.

The chewable composition may additionally contain preservatives, agents that prevent adhesion to oral cavity and crystallization of sugars, flavoring agents, souring agents, coloring agents, or a combination comprising at least one of the foregoing agents. Glycerin, lecithin, hydrogenated palm oil or glyceryl monostearate may be used as a protecting agent of crystallization of the sugars in an amount of about 0.04 wt % to about 10 wt % of the total weight of the ingredients, to prevent adhesion to oral cavity and improve the soft property of the products. Additionally, isomalt or liquid maltitol may be used to enhance the chewing properties of the chewable composition.

The carvedilol or carvedilol subunits can optionally be taste-masked for better patient compliance. Carvedilol may be present in the form of particles, wherein each particle incorporates carvedilol in conjunction with a protective material. The microparticle may be provided as a microcapsule or as a matrix-type microparticle. Microcapsules may incorporate a discrete mass carvedilol surrounded by a discrete, separately observable coating of the protective material. Conversely, in a matrix-type particle, carvedilol is dissolved, suspended or otherwise dispersed throughout the protective material. Certain microparticles may include attributes of both microcapsules and matrix-type particle. For example, a microparticle may incorporate an active agent core incorporating a dispersion of carvedilol in a first protective material and a coating of a second protective material, which may be the same as or different from the first protective material surrounding the active agent core. Alternatively, a microparticle may incorporate an active agent core consisting essentially of carvedilol and a coating incorporating the protective material, the coating itself having some of the carvedilol dispersed within it. Specifically protective material can be a release-retarding material or taste-masking material.

The taste-masked subunits can have a mean outside diameter of up to about 600 micrometers, specifically about 75 to about and 500 micrometers, and more specifically about 150 to about 500 micrometers. Taste-masked subunits above about 200 micrometers may be used. Thus, the taste-masked subunits may be between about 200 mesh and about 30 mesh U.S. standard size, and more specifically between about 100 mesh and about 35 mesh.

Sprinkle dosage forms include controlled-release subunits such as particles or pellets of carvedilol, optionally comprising functional or non-functional coatings, with which a patient or a caregiver can sprinkle the particle/pellet dose into drink or onto soft food. A sprinkle composition may comprise subunits of about 10 to about 100 micrometers in their major dimension. See U.S. Pat. No. 5,084,278, incorporated herein by reference for its teachings regarding microcapsule formulations used for sprinkle dosage forms.

Another suitable oral dosage form is a non-chewable, fast dissolving dosage form. These dosage forms can be made by methods known to those of ordinary skill in the art of pharmaceutical formulations. For example, Cima Labs has produced oral dosage forms including microparticles and effervescents, which rapidly disintegrate in the mouth and provide adequate taste-masking. Cima Labs has also produced a rapidly dissolving dosage form containing the active agent and a matrix that includes a nondirect compression filler and a lubricant. U.S. Pat. No. 5,178,878 and U.S. Pat. No. 6,221,392 provide teachings regarding fast-dissolve dosage forms.

An exemplary fast dissolve dosage form includes a mixture incorporating a water or saliva activated effervescent disintegration agent and subunits such as coated particles. The subunits can include those previously described for the chewable forms. The mixture including the subunits and effervescent disintegration agent may be formulated as a tablet of a size and shape adapted for direct oral administration to a patient. The tablet is substantially completely disintegrable upon exposure to water or saliva. The effervescent disintegration agent is present in an amount effective to aid in disintegration of the tablet, and to provide a distinct sensation of effervescence when the tablet is placed in the mouth of a patient.

The effervescent sensation is not only pleasant to the patient but also tends to stimulate saliva production, thereby providing additional water to aid in further effervescent action. Thus, once the tablet is placed in the patient's mouth, it will disintegrate rapidly and substantially completely without any voluntary action by the patient. Even if the patient does not chew the tablet, disintegration will proceed rapidly. Upon disintegration of the tablet, the subunits are released and can be swallowed as a slurry or suspension. The subunits thus may be transferred to the patient's stomach for dissolution in the digestive tract and systemic distribution of the pharmaceutical ingredient.

The term effervescent disintegration agent includes compounds which evolve gas. The preferred effervescent agents evolve gas by means of chemical reactions which take place upon exposure of the effervescent disintegration agent to water or to saliva in the mouth. The bubble or gas generating reaction is most often the result of the reaction of a soluble acid source and an alkali metal carbonate or carbonate source. The reaction of these two general classes of compounds produces carbon dioxide gas upon contact with water included in saliva.

Such water activated materials may be kept in a generally anhydrous state with little or no absorbed moisture or in a stable hydrated form since exposure to water will prematurely disintegrate the tablet. The acid sources or acid may be those which are safe for human consumption and may generally include food acids, acid anhydrides and acid salts. Food acids include citric acid, tartaric acid, malic acid, fumaric acid, adipic acid, and succinic acids etc. Because these acids are directly ingested, their overall solubility in water is less important than it would be if the effervescent tablet formulations were intended to be dissolved in a glass of water. Acid anhydrides and acid of the above described acids may also be used. Acid salts may include sodium, dihydrogen phosphate, disodium dihydrogen pyrophosphate, acid citrate salts and sodium acid sulfite.

Carbonate sources include dry solid carbonate and bicarbonate salts such as sodium bicarbonate, sodium carbonate, potassium bicarbonate and potassium carbonate, magnesium carbonate and sodium sesquicarbonate, sodium glycine carbonate, L-lysine carbonate, arginine carbonate, amorphous calcium carbonate, or a combination comprising at least one of the foregoing carbonates.

The effervescent disintegration agent is not always based upon a reaction which forms carbon dioxide. Reactants which evolve oxygen or other gasses which are safe are also considered within the scope. Where the effervescent agent includes two mutually reactive components, such as an acid source and a carbonate source, it is preferred that both components react substantially completely. Therefore, an equivalent ratio of components which provides for equal equivalents is preferred. For example, if the acid used is diprotic, then either twice the amount of a mono-reactive carbonate base, or an equal amount of a di-reactive base should be used for complete neutralization to be realized. However, the amount of either acid or carbonate source may exceed the amount of the other component. This may be useful to enhance taste or performance of a tablet containing an overage of either component. In this case, it is acceptable that the additional amount of either component may remain unreacted.

In general, the amount of effervescent disintegration agent useful for the formation of tablets is about 5 wt % to about 50 wt % based on the total weight of the final dosage form, specifically about 15 wt % and about 30 wt %, and more specifically about 20 wt % to about 25 wt %.

Other fast dissolving dosage forms can be prepared without an effervescent agent by using a spray dried carbohydrate or sugar alcohol excipients (e.g. sorbitol, mannitol, xylitol, or a combination comprising at least one of the foregoing, and the like), optionally combined with a disintegrant (e.g. the disintegrant is selected from crospovidone, croscarmellose, sodium starch glycolate, or a combination comprising at least one of the foregoing, and the like), or a glidant (e.g. colloidal silica, silica gel, precipitated silica, or a combination comprising at least one of the foregoing, and the like). Suitable fast-dissolve can be found in U.S. Patent Application Publication US20030118642 A1 to Norman et al. incorporated herein in its entirety.

The tablets of a fast dissolving dosage form typically rapidly disintegrate when orally administered. By “rapid”, it is understood that the tablets disintegrate in the mouth of a patient in less than about 10 minutes, and desirably between about 30 seconds and about 7 minutes, specifically the tablet should dissolve in the mouth between about 30 seconds and about 5 minutes. Disintegration time in the mouth can be measured by observing the disintegration time of the tablet in water at about 37° C. The tablet is immersed in the water without forcible agitation. The disintegration time is the time from immersion for substantially complete dispersion of the tablet as determined by visual observation. As used herein, the term “complete disintegration” of the tablet does not require dissolution or disintegration of the subunits or other discrete inclusions.

Fast-dissolve tablets can be manufactured by well-known tableting procedures. In common tableting processes, the material which is to be tableted is deposited into a cavity, and one or more punch members are then advanced into the cavity and brought into intimate contact with the material to be pressed, whereupon compressive force is applied. The material is thus forced into conformity with the shape of the punches and the cavity. Hundreds, and even thousands, of tablets per minute can be produced in this fashion.

Liquid or semi-solid compositions including emulsions and suspensions may be formulated to provide adequate taste-masking as well as controlled-release properties. A taste-masked liquid composition may comprise a suspension of taste-masked particles (e.g., microparticles). The use of polymeric coatings on the active agent microparticles, which inhibit or retard the rate of dissolution and solubilization of the active agent is one means of overcoming the taste problems with delivery of active agents in suspension. The polymeric coating allows time for all of the particles to be swallowed before the taste threshold concentration is reached in the mouth.

A taste-masked liquid composition thus comprises the active agent, a polymer encapsulating the active agent, and a suspending medium for suspending the encapsulated active agent. The active agent can be taste-masked by the polymer or polymer and suspending medium.

The pharmaceutically active agent or the active agent particle may be suspended, dispersed or emulsified in the suspending medium after encapsulation with the polymer. The suspending medium may be a water-based medium, but may be a non-aqueous carrier as well. The taste-masked liquid composition may further include other optional dissolved or suspended agents to provide stability to the suspension. These include suspending agents or stabilizers such as, for example, methyl cellulose, sodium alginate, xanthan gum, (poly)vinyl alcohol, microcrystalline cellulose, colloidal silicas, bentonite clay, or a combination comprising at least one of the foregoing agents. Other agents used include preservatives such as methyl, ethyl, propyl and butyl parabens, sweeteners such as sucrose, saccharin sodium, aspartame, mannitol, flavorings such as grape, cherry, peppermint, menthol and vanilla flavors, and antioxidants or other stabilizers, or a combination comprising at least one of the foregoing agents.

Encapsulation of the microparticle or active agent particle by the polymer may be performed by a method such as suspending, dissolving, or dispersing in a solution or dispersion of polymer coating material and spray drying, fluid-bed coating, simple or complex coacervation, coevaporation, co-grinding, melt dispersion and emulsion-solvent evaporation techniques, and the like.

The polymer coated carvedilol can be prepared into a reconstitutable powder, ie., a dry powder active agent product that is reconstituted as suspensions or emulsions in a liquid vehicle such as water prior to usage. The reconstitutable powders have a long shelf life and the suspensions, once reconstituted, have adequate taste-masking.

The controlled-release carvedilol compositions may be prepared into solid dosage forms for oral administration including, for example, a tablet, a tablet in capsule, a plurality of subunits in capsule, a plurality of subunits in a tablet, a chewable tablet, a gummy form, a candy form, a sprinkle, a taste-masked composition, or a fast dissolve tablet. In such solid dosage forms, the carvedilol may be admixed with a pharmaceutically acceptable excipient to aid in processing, etc. As used herein, “pharmaceutically acceptable excipient” means any other component added to the pharmaceutical composition other than the active agent. Excipients may be added to facilitate manufacture, enhance stability, control release, enhance product characteristics, enhance bioavailability, enhance patient acceptability, etc. Pharmaceutical excipients include carriers, fillers, binders, disintegrants, lubricants, glidants, compression aids, colors, sweeteners, preservatives, suspending agents, dispersing agents, film formers, flavors, printing inks, etc.

Binders hold the ingredients in the composition together. Exemplary binders include, for example, polyvinyl pyrrolidone, hydroxypropyl cellulose, hydroxypropyl methylcellulose, methylcellulose and hydroxyethyl cellulose, sugars, or a combination comprising at least one of the foregoing binders.

Disintegrants expand when wet causing a tablet or other dosage form to break apart. Exemplary disintegrants include water swellable substances, for example, low-substituted hydroxypropyl cellulose, e.g. L-HPC; cross-linked polyvinyl pyrrolidone (PVP-XL), e.g. Kollidon® CL and Polyplasdone® XL; cross-linked sodium carboxymethylcellulose (sodium croscarmellose), e.g. Ac-di-sol®, Primellose®; sodium starch glycolate, e.g. Primojel®; sodium carboxymethylcellulose; sodium carboxymethyl starch, e.g. Explotab®; ion-exchange resins, e.g. Dowex® or Amberlite®; microcrystalline cellulose, e.g. Avicel®; starches and pregelatinized starch, e.g. Starch 1500®; formalin-casein, or a combination comprising at least one of the foregoing water swellable substances.

Lubricants, for example, aid in the processing of powder materials. Exemplary lubricants include calcium stearate, glycerol behenate, magnesium stearate, mineral oil, polyethylene glycol, sodium stearyl fumarate, stearic acid, talc, vegetable oil, zinc stearate, or a combination comprising at least one of the foregoing lubricants. Glidants include, for example, silicon dioxide.

Certain compositions described herein contain a filler, such as a water insoluble filler, water soluble filler, or a combination comprising at least one of the foregoing. The filler may be a water insoluble filler, such as silicon dioxide, titanium dioxide, talc, alumina, starch, kaolin, polacrilin potassium, powdered cellulose, microcrystalline cellulose, sodium citrate, dicalcium phosphate or a combination comprising at least one of the foregoing fillers. Exemplary water-soluble fillers include water soluble sugars and sugar alcohols, specifically lactose, glucose, fructose, sucrose, mannose, dextrose, galactose, the corresponding sugar alcohols and other sugar alcohols, such as mannitol, sorbitol, xylitol, or a combination comprising at least one of the foregoing fillers.

Optionally, certain compositions described herein may be coated with a non-functional coating, or multiple functional or non-functional coatings. By “functional coating” is meant to include a coating that modifies the release properties of the total composition, for example, a sustained-release or delayed-release coating. By “non-functional coating” is meant to include a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition.

Also included herein are pharmaceutical kits comprising one or a plurality of containers containing the controlled-release carvedilol dosage forms as described herein. The kits may further comprise one or more conventional pharmaceutical kit components, such as, for example, one or more containers to aid in facilitating compliance with a particular dosage regimen; one or more carriers; printed instructions, either as inserts or as labels, indicating quantities of the components to be administered, or guidelines for administration. Exemplary kits can be in the form of bubble or blister pack cards, optionally arranged in a desired order for a particular dosing regimen. Suitable blister packs that can be arranged in a variety of configurations to accommodate a particular dosing regimen are well known in the art or easily ascertained by one of ordinary skill in the art.

Those forms existing as liquids, solutions, emulsions, or suspensions can be packaged for convenient dosing of geriatric patients. For example, prefilled droppers (such as eye droppers or the like), prefilled syringes, and similar containers housing the liquid, solution, emulsion, or suspension form of the controlled-release forms are contemplated.

“Bioavailability” means the extent or rate at which an active agent is absorbed into a living system or is made available at the site of physiological activity. For active agents that are intended to be absorbed into the bloodstream, bioavailability data for a given composition may provide an estimate of the relative fraction of the administered dose that is absorbed into the systemic circulation. “Bioavailability” can be characterized by one or more pharmacokinetic parameters.

“Pharmacokinetic parameters” describe the in vivo characteristics of an active agent (or surrogate marker for the active agent) over time, such as plasma concentration (C), Cₘₐₓ, Cₙ, C₂₄, Tₘₐₓ, and AUC. “Cₘₐₓ” is the measured concentration of the active agent in the plasma at the point of maximum concentration. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “AUC” is the area under the curve of a graph of the measured concentration of an active agent (typically plasma concentration) versus time, measured from one time point to another time point. For example AUC₀₋ₜ is the area under the curve of plasma concentration versus time from time 0 to time t. The AUC_{0-∞} or AUC_{0-INF} is the calculated area under the curve of plasma concentration versus time from time 0 to time infinity.

Bioavailability can be determined for a carvedilol composition under different administration conditions e.g., non-fasted versus fasted. Exemplary study considerations can be found in the Federal Drug Administration's (FDA) guidelines and criteria, including “Guidance for Industry, Food-Effect Bioavailability and Fed Bioequivalence Studies” available from the U.S. Department of Health and Human Services (DHHS), Food and Drug Administration (FDA), Center for Drug Evaluation and Research (CDER) December 2002, incorporated herein in its entirety.

“Food” typically means a solid food or mixed solid/liquid food with sufficient bulk and fat content that it is not rapidly dissolved and absorbed in the stomach. In one embodiment, food means a meal, such as breakfast, lunch or dinner. The terms “taken with food”, “fed” and “non-fasted” are equivalent and are as given by FDA guidelines and criteria. In one embodiment, “with food” means that the dosage form is administered to a patient between about 30 minutes prior to about 2 hours after eating a meal. In another embodiment, with food means that the dosage form is administered at substantially the same time as the eating the meal.

The terms “without food”, “fasted” and “an empty stomach” are equivalent and are as given by FDA guidelines and criteria. In one embodiment, fasted means the condition wherein no food is consumed within 1 hour prior to administration of the dosage form or 2 hours after administration of the dosage form. In another embodiment, fasted means the condition wherein no food is consumed within 1 hour prior to administration of the dosage form to 2 hours after administration of the dosage form.

“Bioequivalence” means the absence of a significant difference in the rate and extent to which the active agent or surrogate marker for the active agent in pharmaceutical equivalents or pharmaceutical alternatives becomes available at the site of action when administered in an appropriately designed study.

In one embodiment, bioequivalence is any definition thereof as promulgated by the U.S. Food and Drug Administration or any successor agency thereof. In a specific embodiment, bioequivalence is determined according to the Federal Drug Administration's (FDA) guidelines and criteria, including “GUIDANCE FOR INDUSTRY BIOAVAILABILITY AND BIOEQUVALENCE STUDIES FOR ORALLY ADMINISTERED DRUG PRODUCTS-GENERAL CONSIDERATIONS” available from the U.S. Department of Health and Human Services (DHHS), Food and Drug Administration (FDA), Center for Drug Evaluation and Research (CDER) March 2003 Revision 1; and “GUIDANCE FOR INDUSTRY STATISTICAL APPROACHES TO ESTABLISHING BIOEQUIVALENCE” DHHS, FDA, CDER, January 2001, both of which are incorporated herein in their entirety.

In another embodiment, bioequivalence is determined according to the European Medicines Agency (EMEA) document “Note for Guidance on the Investigation of Bioavailability and Bioequivalence”, issued Jul. 26, 2001, available from EMEA.

“Reference drug” means the carvedilol product as described in U.S. Federal Food and Drug Administration's New Drug Application No. 022012 approved on Oct. 20, 2006 (strengths of 10, 20, 40, and 80 mg carvedilol phosphate (1:1) hemihydrate) and by its brand name COREG CR™ carvedilol phosphate controlled-release capsules. COREG CR™ is designed for once-a-day administration and is said to provide 24-hour efficacy to minimize blood pressure variability. The extended-release hard gelatin capsules contain carvedilol phosphate immediate-release and controlled-release microparticles that are drug-layered and then coated with methacrylic acid copolymers. Inactive ingredients are crospovidone, hydrogenated castor oil, hydrogenated vegetable oil, magnesium stearate, methacrylic acid copolymers, microcrystalline cellulose, and povidone.

“Reference drug, immediate release” means a carvedilol product as described in U.S. Federal Food and Drug Administration's New Drug Application No. 020297 approved on May 29, 1997 (3.125 mg) and Sep. 14, 1995 (6.25 mg, 12.5 mg, and 25 mg) as provided in the U.S. Federal Food and Drug Administration's Orange Book, Approved Drug Products with Therapeutic Equivalence Evaluations. COREG® is a capsule product which is marketed by GlaxoSmithKline. COREG® 12.5 mg strength is the “reference listed drug” under 21 CFR 314.94(a)(3)), i.e., the listed drug identified by FDA as the drug product upon which an applicant relies in seeking approval of its ANDA.

In one embodiment, the controlled-release carvedilol composition is bioequivalent to the Reference drug capsule form (COREG CR™, New Drug Application No. 022012).

In one embodiment, the controlled-release carvedilol composition is bioequivalent to the Reference drug, immediate release capsule form (COREG®, New Drug Application No. 020297) at a twice daily dosing.

In an embodiment, bioequivalence of carvedilol composition to a reference drug is determined by an in vivo bioequivalence study to determine a pharmacokinetic parameter for the carvedilol composition. Specifically, bioequivalence can be determined by an in vivo bioequivalence study comparing a pharmacokinetic parameter for the two compositions. A pharmacokinetic parameter for the carvedilol composition or the reference drug can be measured in a single or multiple dose bioequivalence study using a replicate or a nonreplicate design. For example, the pharmacokinetic parameters for a carvedilol composition of the present invention and for a reference drug can be measured in a single dose bioequivalence study using a two-period, two-sequence crossover design. Alternately, a four-period, replicate design crossover study may also be used. Single doses of the test composition and reference drug are administered and blood or plasma levels of the active agent are measured over time. Pharmacokinetic parameters characterizing rate and extent of active agent absorption are evaluated statistically.

The area under the plasma concentration-time curve from time zero to the time of measurement of the last quantifiable concentration (AUC₀₋ₜ) and to infinity (AUC_{0-∞}), Cₘₐₓ, and Tₘₐₓ can be determined according to standard techniques. Statistical analysis of pharmacokinetic data is performed on logarithmic transformed data (e.g., AUC₀₋ₜ, AUC_{0-∞}, or Cₘₐₓ data) using analysis of variance (ANOVA).

In some embodiments a single dose pharmacokinetic study is performed under non-fasted or fasted conditions.

In other embodiments, the single dose pharmacokinetic study is conducted between the controlled-release carvedilol composition and the reference listed drug using the strength specified by the FDA in APPROVED DRUG PRODUCTS WITH THERAPEUTIC EQUIVALENCE EVALUATIONS(ORANGE BOOK).

Under U.S. FDA guidelines, two products (e.g. an inventive composition and COREG® or COREG CR™) or methods (e.g., dosing under non-fasted versus fasted conditions) are bioequivalent if the 90% Confidence Interval (CI) limits for a ratio of the geometric mean of logarithmic transformed AUC_{0-∞}, AUC₀₋ₜ, and Cₘₐₓ for the two products or two methods are about 0.80 to about 1.25.

To show bioequivalence between two compounds or administration conditions pursuant to Europe's EMEA guidelines, the 90% CI limits for a ratio of the geometric mean of logarithmic transformed AUC_{0-∞} and AUC₀₋ₜ for the two products or methods are about 0.80 to about 1.25. The 90% CI limits for a ratio of the geometric mean of logarithmic transformed Cₘₐₓ for the two products or methods can have a wider acceptance range when justified by safety and efficacy considerations. For example the acceptance range can be about 0.70 to about 1.43, specifically about 0.75 to about 1.33, and more specifically about 0.80 to about 1.25.

In one embodiment, in a given experiment, a carvedilol composition is considered to be bioequivalent to COREG® (dosed BID) or COREG CR™ if both the Test/Reference ratio for the geometric mean of logarithmic transformed AUC_{0-∞}, AUC₀₋ₜ, or Cₘₐₓ ratio along with its corresponding lower and upper 90% CI limits are within a lower limit of about 0.80 and an upper limit of about 1.25. Thus, for direct comparison between a carvedilol composition and COREG® or COREG CR™, it is sometimes preferred to determine the pharmacokinetic parameters for the carvedilol composition and COREG® or COREG CR™ side-by side in the same pharmacokinetic study.

In one embodiment, the geometric mean of logarithmic transformed AUC_{0-∞} of the controlled-release composition is within about 80% and about 125% of 525 ng·h/ml; or the geometric mean of logarithmic transformed Cₘₐₓ of the controlled-release composition is within about 70% and about 143% of 54 ng/ml, specifically within about 80% and about 125% of 54 ng/ml.

In one embodiment, the controlled-release composition exhibits a single phase release (single Tₘₐₓ) within the first 10 hours after oral administration to a patient, specifically within the first 7 hours, more specifically within the first 5.5 hours.

In another embodiment, the controlled-release composition such that the controlled-release composition exhibits a dual phase release having a first peak plasma Tₘₐₓ₁ of greater than 4 hours, specifically greater than about 5 hours, more specifically greater than about 5.5 hours, and yet more specifically greater than about 6 hours; and a second peak plasma Tₘₐₓ₂ of greater than 10 hours, specifically greater than about 11 hours, and more specifically greater than about 11.5 hours.

In yet another embodiment, the controlled-release composition releases about 30% of carvedilol within the first 10 hours after oral administration to a patient.

In one embodiment, a carvedilol controlled-release composition exhibits a ratio of a geometric mean of logarithmic transformed AUC_{0-∞} of the composition to a geometric mean of logarithmic transformed AUC_{0-∞} of reference drug (New Drug Application No. 022012) of about 0.80 to about 1.25.

In another embodiment, a carvedilol controlled-release composition exhibits a ratio of a geometric mean of logarithmic transformed AUC₀₋ₜ of the composition to a geometric mean of logarithmic transformed AUC₀₋ₜ of reference drug (New Drug Application No. 022012) of about 0.80 to about 1.25.

In yet another embodiment, a carvedilol controlled-release composition exhibits a ratio of a geometric mean of logarithmic transformed Cₘₐₓ of the composition to a geometric mean of logarithmic transformed Cₘₐₓ of reference drug (New Drug Application No. 022012) of about 0.70 to about 1.43.

In yet another embodiment, a carvedilol controlled-release composition exhibits a ratio of a geometric mean of logarithmic transformed Cₘₐₓ of the composition to a geometric mean of logarithmic transformed Cₘₐₓ of reference drug (New Drug Application No. 022012) of about 0.80 to about 1.25.

In one embodiment, the controlled-release carvedilol composition, when administered under fasted conditions is bioequivalent to the same controlled-release carvedilol composition administered under non-fasted conditions when the 90% Confidence Interval (CI) limits for a ratio of the geometric mean of logarithmic transformed AUC_{0-∞}, AUC₀₋ₜ, or Cₘₐₓ for the two administration conditions are about 0.80 to about 1.25.

A dissolution profile is a plot of the cumulative amount of active agent released as a function of time. A dissolution profile can be measured utilizing the Drug Release Test <724>, which incorporates standard test USP 26 (Test <711>). A profile is characterized by the test conditions selected such as, for example, apparatus type, shaft speed, temperature, volume, and pH of the dissolution medium. More than one dissolution profile may be measured. For example, a first dissolution profile can be measured at a pH level approximating that of the stomach, and a second dissolution profile can be measured at a pH level approximating that of one point in the intestine or several pH levels approximating multiple points in the intestine.

A highly acidic pH may be employed to simulate the stomach and a less acidic to basic pH may be employed to simulate the intestine. By the term “highly acidic pH” is meant a pH of about 1 to about 4.5. A pH of about 1.2, for example, can be used to simulate the pH of the stomach. By the term “less acidic to basic pH” is meant a pH of greater than about 4 to about 7.5, specifically about 6 to about 7.5. A pH of about 6 to about 7.5, specifically about 6.8, can be used to simulate the pH of the intestine.

The controlled-release composition may be tested using a USP Type II apparatus, at 50 rpm, 900 mL of media selected from the group of purified water, acidic buffer of pH 4.5, 0.1 N HCl and pH 6.8 phosphate buffer.

The controlled-release composition may be tested sequentially using a USP Type II apparatus, at 50 rpm, 900 mL of acidic media up to pH 4.5, then using a USP Type II apparatus, at 75 rpm, more specifically at 100 rpm and 900 mL of pH 6.8 phosphate buffer, where the controlled-release composition exhibits not more than about 80% carvedilol released in 6 hours; and not less than about 80% carvedilol released in 18 hours.

Carvedilol exhibits low solubility in aqueous media, especially in media of increasingly basic pH. Accordingly, it would be beneficial to prepared microparticles or nanoparticles of carvedilol as a way of increasing its solubility, and perhaps increase its in vivo bioavailability. Accordingly, the preparation of microparticles and nanoparticles of carvedilol is contemplated herein.

In one embodiment, a composition comprises carvedilol and a surfactant, wherein the carvedilol/surfactant particle has a mean diameter of about 1 to about 500 micrometers, specifically about 5 to about 250 micrometers, and more specifically about 25 to about 100 micrometers.

Any conventional means of measuring particle size can be used, for example light scattering techniques.

To prepare micrometer sized carvedilol particles, carvedilol is micronized with a surfactant using a jet mill micronizer. Suitable processes to prepare micrometer sized active agent particles can be found in U.S. Pat. No. 4,895,726.

Exemplary surfactants include amphoteric, non-ionic, cationic or anionic surfactants. Particular examples include sodium lauryl sulfate, monooleate, monolaurate, monopalmitate, monostearate or another ester of polyoxyethylene sorbitane, sodium dioctylsulfosuccinate, lecithin, stearylic alcohol, cetostearylic alcohol, cholesterol, polyoxyethylene ricin oil, polyoxyethylene fatty acid glycerides, Poloxamer®, or a combination comprising at least one of the forgoing surfactants.

The nanoparticulate compositions comprise carvedilol and a surface stabilizer adsorbed on the surface of the carvedilol. The carvedilol/surface stabilizer particle can have a mean diameter of less than about 1 micrometer, specifically about 5 to about 800 nanometers, more specifically about 25 to about 600 nanometers, and yet more specifically about 50 to 400 nanometers.

Suitable surface stabilizers include nonionic and anionic surfactants, gelatin, casein, lecithin (phosphatides), gum acacia, tragacanth, stearic acid, benzalkonium chloride, calcium stearate, glyceryl monostearate, cetostearl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium lauryl sulfate (a.k.a. sodium dodecylsulfate), carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, and polyvinylpyrrolidone.

Methods of making carvedilol nanoparticles include those found in U.S. Pat. No. 5,145,684. Exemplary methods of preparing carvedilol nanoparticle include wet milling a dispersion of carvedilol in a liquid dispersion medium in the presence of grinding media, optionally in the presence of a surface stabilizer. The surface stabilizer may be added after milling rather than comilled with the active agent. Suitable mills include a ball mill, an attritor mill, a vibratory mill a sand mill, a bead mill, and the like.

In one embodiment, the carvedilol can be in amorphous form. Such amorphous forms can be beneficial for increasing the solubility of the active agent or bioavailability. Various methods of preparing amorphous carvedilol include coprocessing the active agent with a complexing agent to result in a solid dispersion. The complexing agent aids to retain the active agent in amorphous form and to retard crystallization. The solid dispersion can be prepared by a melt process or solution process.

The melt method involves melting the active agent and complexing agent to temperatures sufficient to achieve a flowable, molten mass. The melt is quickly cooled and processed into particulate form by grinding, and the like. The cooling process can include spray congealing (chilling), and the like. The temperature and choice of complexing agent can be made such that there is no degradation of the active agent or complexing agent during the process.

In one embodiment, the complexing agent is melted and the active agent is added to the melt and subsequently dissolved or transformed into amorphous form. The resulting melt is then cooled to form the solid dispersion.

The solution process involves dissolving the active agent and complexing agent in a solvent system, followed by removal of the solvent to leave a solid dispersion of amorphous carvedilol and complexing agent. Such a process can include preparation of a separate solution of active agent and solvent system which is then blended with a solution or suspension of complexing agent and solvent system, followed by removal of the solvent. Other approaches include dissolving the active agent and complexing agent in the same solution. Optionally, to aid in solubilization, the solutions or suspensions can be heated.

Suitable complexing agents include a polyethylene glycol (e.g., PEG 200, PEG 300, PEG 400, PEG 540, PEG 600, PEG 900, PEG 1000, PEG 1450, PEG 1540, PEG 2000, PEG 3000, PEG 3350, PEG 4000, PEG 4600, PEG 8000, and the like), polyoxyethylene alkyl ethers, polyoxyethylene sorbitan fatty acid esters, polyethylene oxide, polyvinylpyrrolidone, polyvinyl alcohols, or a combination comprising at least one of the foregoing complexing agents.

Suitable solvents for the solvent system include water, lower alkyl alcohols (e.g., methanol, ethanol, n-propanol, isopropanol, etc.), lower alkyl ketones or acetates (e.g., acetone, ethyl acetate, etc.), lower alkyl ethers (e.g., ethyl ether, tetrahydrofuran, etc.), acetonitrile, lower halogenated alkyls (e.g., dichloromethane, etc.), or a combination comprising at least one of the foregoing solvents. Choice and amount of solvent is dependant upon the choice of polymer and carvedilol salt.

The solvent can be removed by any variety of methods such as evaporation optionally under reduced pressure or under heat, precipitation by a non-solvent, freeze drying, spray drying, and the like.

The amount of active agent and complexing agent can be about 1:20 to about 5:1, specifically 1:10 to about 1:1, and yet more specifically about 1:5 to about 1:3 active agent to complexing agent wt/wt.

In addition to the complexing agent, the solid dispersion can further comprise other excipients to aid in processing such as surfactants, plasticizers, fillers, and the like.

In another embodiment, a solution of carvedilol and optional complexing agent can be spray dried onto a carrier to result in an amorphous carvedilol complex. The presence of the carrier or complexing agent prevents crystallization of the carvedilol. The complexing agents previously discussed may be used in this embodiment, specifically the polyethylene glycols. Acceptable solvents for use in this embodiment include those previously discussed above. Suitable carriers include cellulosic polymers such as an alkylcelluloses (e.g., methylcellulose, ethylcellulose, and the like) modified celluloses such as a carboxymethylcellulose, a hydroxypropylmethylcellulose, a crosslinked sodium carboxymethylcellulose, a hydroxyl alkylcellulose (e.g., hydroxypropylcellulose); polyvinylpyrrolidone, a polyvinyl alcohol, a polysaccharide, a mono or disaccharide (e.g., lactose), a sugar alcohol, other pharmaceutically acceptable polymer excipients, or a combination comprising at least one of the foregoing carriers. Suitable spray drying techniques are known in the art.

Determination of the extent of conversion of the crystalline form of the active agent to an amorphous form can be determined using analytical techniques known in the art, including x-ray diffraction analysis, differential scanning calorimetry, and the like.

In one embodiment, the solid dispersion comprises an amorphous carvedilol salt. The amorphous carvedilol salt can be amorphous carvedilol phosphate salt, specifically carvedilol hydrogen phosphate, carvedilol dihydrogen phosphate, a hydrate of the foregoing, solvate of the foregoing, or a combination comprising at least one of the foregoing salts. In a particular embodiment, the solid dispersion comprises carvedilol dihydrogen phosphate, hemihydrate.

The following examples further illustrate the invention but, of course, should not be construed as in any way limiting its scope.

### EXAMPLES

### Example 1

### Immediate-Release Subunits: Compression

Immediate-release subunits can be prepared by a granulation process followed by compression into compressed subunits. All amounts of the components in Table 1 are provided as weight percent.

TABLE 1

| Ingredient | A1 | B1 | C1 | D1 | E1 | F1 | G1 | H1 | I1 |
|---|---|---|---|---|---|---|---|---|---|
| Carvedilol dihydrogen | 40.0-42.0 | 40.0-42.0 | 40.0-42.0 | 40.0-42.0 | 40.0-42.0 | 20.0-21.0 | 20.0-21.0 | 20.0-21.0 | 20.0-21.0 |
| phosphate, | | | | | | | | | |
| hemihydrate | | | | | | | | | |
| Microcrystalline | 53.0-55.0 | 43.0-45.0 | 43.0-45.0 | 43.0-45.0 | 43.0-45.0 | 34.0-38.0 | 34.0-35.0 | 34.0-35.0 | 34.0-35.0 |
| cellulose (Avicel PH | | | | | | | | | |
| 101) | | | | | | | | | |
| Lactose monohydrate | — | — | — | — | — | 32.0-34.0 | 32.0-34.0 | 32.0-34.0 | 32.0-34.0 |
| (Fast-Flo) | | | | | | | | | |
| Croscarmellose | 5.0 | 5.0 | 5.0 | 5.0 | 5.0 | — | — | — | — |
| Crospovidone | — | — | — | — | — | 10.0 | 10.0 | 10.0 | 10.0 |
| (Polyplasdone XL10) | | | | | | | | | |
| Hydroxypropylmethyl | — | 10.0 | — | — | — | — | 3.0 | — | — |
| cellulose | | | | | | | | | |
| Polyvinylpyrrolidone | — | — | 10.0 | — | — | — | — | 3.0 | — |
| Hydroxypropyl | — | — | — | 10.0 | — | — | — | — | 3.0 |
| cellulose | | | | | | | | | |
| Methylcellulose | — | — | — | — | 10.0 | — | — | — | — |
| Total | 100.0 | 100.0 | 100.0 | 100.0 | 100.0 | 100.0 | 100.0 | 100.0 | 100.0 |

The components of Formulation A1 are dry mixed in a high shear granulator to form a mixture which is then compressed into small tablets (mean diameter of about 3 to about 7 mm) or microtablets (mean diameter of about 0.7 to about 1.4 mm). The components of Formulations B1-E1 are each independently wet granulated with water or other suitable solvent in a high shear granulator to form a wet granulation mixture. The wet granulation mixture is screened, dried, and milled. Each of the resulting blends is compressed into small tablets or microtablets, each containing about 1 to about 10 mg of carvedilol salt per subunit depending upon the size of the subunit. If necessary, small amounts of lubricant (e.g. magnesium stearate) or anticaking compounds (silicon dioxide) can be added to the compressible mixtures prior to compression.

### Example 2

### Controlled-Release Subunits: Compression

Controlled-release subunits can be prepared by a granulation process followed by compression into compressed subunits. All amounts of the components in Table 2 are provided as weight percent.

TABLE 2

| Ingredient | A2 | B2 | C2 | D2 | E2 | F2 | G2 | H2 |
|---|---|---|---|---|---|---|---|---|
| Carvedilol dihydrogen | 30.0 | 30.0 | 30.0 | 30.0 | 30.0 | 30.0 | 30.0 | 30.0 |
| phosphate, hemihydrate | | | | | | | | |
| Ethyl cellulose T-10 | 58.0 | 58.0 | 58.0 | 58.0 | 48.0 | 48.0 | 48.0 | 48.0 |
| Lactose monohydrate | 10.0 | 10.0 | 10.0 | 10.0 | 10.0 | 10.0 | 10.0 | 10.0 |
| (Fast-Flo) | | | | | | | | |
| Polyethylene glycol | 2.0 | — | — | — | 2.0 | 2.0 | 2.0 | 2.0 |
| Poloxamer | — | 2.0 | — | — | — | — | — | — |
| Dibutyl phthalate | — | — | 2.0 | — | — | — | — | — |
| Polysorbate | — | — | — | 2.0 | | — | — | — |
| Hydroxypropylmethyl | — | — | — | — | 10.0 | — | — | — |
| cellulose | | | | | | | | |
| Polyvinylpyrrolidone | — | — | — | — | — | 10.0 | — | — |
| Hydroxypropyl cellulose | — | — | — | — | — | — | 10.0 | — |
| Methyl cellulose | — | — | — | — | — | — | — | 10.0 |
| Total | 100.0 | 100.0 | 100.0 | 100.0 | 100.0 | 100.0 | 100.0 | 100.0 |

The components of Formulation A2-D2 are each independently dry mixed in a high shear granulator to form a mixture which is then compressed into small tablets or microtablets. The components of Formulations E2-H2 are each independently wet granulated with water or other suitable solvent in a high shear granulator to form a wet granulation mixture. The wet granulation mixture is screened, dried, and milled. Each of the resulting blends is compressed into small tablets or microtablets. If necessary, small amounts of lubricant can be added to the compressible mixtures prior to compression.

### Example 3

### Controlled-Release Subunits: Compression

Controlled-release subunits can be prepared by a granulation process followed by compression into compressed subunits. All amounts of the components in Table 3 are provided as weight percent.

TABLE 3

| Ingredient | A3 | B3 | C3 | D3 | E3 | F3 | G3 | H3 |
|---|---|---|---|---|---|---|---|---|
| Carvedilol dihydrogen | 30.0 | 30.0 | 30.0 | 30.0 | 30.0 | 30.0 | 30.0 | 30.0 |
| phosphate, hemihydrate | | | | | | | | |
| Eudragit L-100/S-100 1:1 | 58.0 | 58.0 | 58.0 | 58.0 | 48.0 | 48.0 | 48.0 | 48.0 |
| wt/wt mixture | | | | | | | | |
| Lactose monohydrate | 10.0 | 10.0 | 10.0 | 10.0 | 10.0 | 10.0 | 10.0 | 10.0 |
| (Fast-Flo) | | | | | | | | |
| Polyethylene glycol | 2.0 | — | — | — | 2.0 | 2.0 | 2.0 | 2.0 |
| Poloxamer | — | 2.0 | — | — | — | — | — | — |
| Dibutyl phthalate | — | — | 2.0 | — | — | — | — | — |
| Polysorbate | — | — | — | 2.0 | | — | — | — |
| Hydroxypropylmethyl | — | — | — | — | 10.0 | — | — | — |
| cellulose | | | | | | | | |
| Polyvinylpyrrolidone | — | — | — | — | — | 10.0 | — | — |
| Hydroxypropyl cellulose | — | — | — | — | — | — | 10.0 | — |
| Methyl cellulose | — | — | — | — | — | — | — | 10.0 |
| Total | 100.0 | 100.0 | 100.0 | 100.0 | 100.0 | 100.0 | 100.0 | 100.0 |

The components of Formulation A3-D3 are each independently dry mixed in a high shear granulator to form a mixture which is then compressed into small tablets or microtablets. The components of Formulations E3-H3 are each independently wet granulated with water or other suitable solvent in a high shear granulator to form a wet granulation mixture. The wet granulation mixture is screened, dried, and milled. Each of the resulting blends is compressed into small tablets or microtablets. If necessary, small amounts of lubricant can be added to the compressible mixtures prior to compression.

### Example 4

### Controlled-Release Subunits: Extrusion/Spheronization

The subunits can be prepared as controlled-release pellets. The formulations of Examples 2 and 3 can be prepared into spherical pellets using extrusion/spheronization techniques well known in the pharmaceutical art. If necessary, small amounts of the release-retarding matrix material or the binder can be replaced with microcrystalline cellulose to aid in the spheronization process.

### Example 5

### Subunits with a Controlled-Release Coating; Copolymer of Acrylic and Methacrylic Acid

The immediate-release or controlled-release subunits of Examples 1-4 can be coated with a release-retarding coating material to provide controlled-release subunits. Table 4 provides a coating formulation based on copolymers of acrylic and methacrylic acid, all amounts are in weight percent.

TABLE 4

| Ingredient | A5 | B5 | C5 | D5 | E5 | F5 | G5 | H5 |
|---|---|---|---|---|---|---|---|---|
| Eudragit ® L30-D-55 | 74-75 | — | — | — | 57-58 | — | — | — |
| Eudragit ® L100 | — | 74-75 | — | — | — | 55-56 | — | — |
| Eudragit ® RS/RL 90/10 | — | — | 74-75 | 72.5-73 | — | — | 57-58 | — |
| Eudragit ® S-100 | — | — | — | — | — | — | — | 55-56 |
| Magnesium stearate | — | — | | 2-2.5 | — | — | — | — |
| Polyethylene Glycol 600 | 5-6 | 5-6 | 5-6 | 5-6 | — | — | — | — |
| Triethyl citrate | | | | | 6-7 | 10-11 | 6-7 | 10-11 |
| Talc | 19-20 | 19-20 | 19-20 | 19-20 | 35-36 | 33-34 | 35-36 | 33-34 |
| Water/Ethyl Alcohol* | | | | | | | | |

*Removed in process

Polyethylene glycol or triethyl citrate is added to a water or ethyl alcohol dispersion of a copolymer of acrylic and methacrylic acid and mixed. Talc is added while stirring with a propeller mixer. The uncoated subunits are added into a perforated coating pan or a fluid bed with a Wurster insert. The coating is sprayed onto the subunits. A coating level of about 5-25 weight % coat weight is applied to the immediate-release subunits of Example 1 or the controlled-release subunits of Examples 2-4. Optionally, a coating level of about 1-10 weight % of Opadry Clear is applied to the subunits and dried prior to the coating of copolymer of acrylic and methacrylic acid. The coated subunits are filled into capsule shells.

### Example 6

### Subunits with a Controlled-Release Coating; Ethyl Cellulose

The immediate-release or controlled-release subunits of Examples 1-2 can be coated with a release-retarding coating material to provide controlled-release subunits. Table 5 provides a coating formulation based on ethyl cellulose, all amounts are in weight percent.

TABLE 5

| Ingredient | A6 | B6 | C6 |
|---|---|---|---|
| Surelease ® (E-7-19010) | 100 | 90 | 80 |
| Opadry Clear (YS-3-7011) | — | 10 | 20 |
| Water* | | | |

*Removed in process

The uncoated subunits are added into a perforated coating pan or a fluid bed with a Wurster insert. The coating is sprayed onto the subunits. A coating level of about 5-25% coat weight is applied to the immediate-release subunits of Example 1 or the controlled-release subunits of Examples 2-4. Optionally, a coating level of about 1-10 weight % of Opadry Clear is applied to the subunits and dried prior to the coating of Surelease®. The coated subunits are filled into capsule shells.

### Example 7

### Dissolution Profiles for Coated Subunits

Exemplary controlled-release subunits are prepared from the immediate-release subunits of Example 1 and the controlled-release coatings of 5 and 6. A number of tablets equaling 40 mg of carvedilol dihydrogen phosphate, hemihydrate are taken from each sample and tested in the following dissolution media: 0.1 N HCl, pH4.5 acetate buffer, and pH6.8 phosphate buffer. The dissolution conditions are 900 ml of dissolution medium, USP apparatus 2, paddle speed 50 rpm, temperature of 37° C.±2° C.

The immediate-release subunits of Example 1, formulation F1 are coated with a 3 weight % coating of Opadry Clear, dried and coated with 14-22 weight % of the Surelease®/Opadry Clear 80:20. The resulting controlled-release subunits exhibit a release profile accordingly as percent released shown in Table 6.

TABLE 6

| Time | | Acetate buffer | Phosphate |
|---|---|---|---|
| (hours) | 0.1 N HCl | pH 4.5 | buffer pH 6.8 |
| 2 | 2-52 | 3-51 | 13-38 |
| 4 | 35-66 | 37-64 | 37-60 |
| 6 | 49-72 | 60-66 | 40-64 |
| 8 | 57-77 | 67-73 | 49-66 |
| 10 | 68-85 | 72-88 | 58-69 |

The immediate-release subunits of Example 1, formulation F1 are coated with a 3 weight % coating of Opadry Clear, dried and coated with 6-20 weight % of the Eudragit L30D55 coating of Example 5, formulation E5. The resulting controlled-release subunits exhibit a release profile accordingly as percent released shown in Table 7.

TABLE 7

| Time | | Phosphate |
|---|---|---|
| (hours) | 0.1 N HCl | buffer pH 6.8 |
| 2 | 0 | 32-50 |
| 4 | 0 | 37-56 |
| 6 | 0 | 38-58 |
| 8 | 0 | 40-59 |
| 10 | 0 | 43-60 |

### Example 8

### Controlled-Release Carvedilol Capsules

The controlled-release subunits of Example 7 are combined with immediate release subunits of Example 1, loaded in a gelatin capsule to result in a controlled-release carvedilol capsule containing a total of about 40 mg of carvedilol hydrogen phosphate per capsule. The controlled-release carvedilol capsule when tested under dissolution conditions of 900 ml of dissolution medium, USP apparatus 2, paddle speed 50 rpm, temperature of 37° C.±2° C. exhibits a release of about 10 to about 80 wt. % of the total amount of carvedilol is released after 7 hours when the dissolution medium has a pH of about 0.1 to about 4.5 (e.g. 0.1 N HCl, pH 4.5 acetate buffer; and about 80 to about 100 wt. % of the total amount of carvedilol is released after 7 hours when the dissolution medium has a pH of greater than 4.5 (e.g., phosphate buffer, deionized water).

### Example 9

### Controlled-Release Carvedilol Capsules

Controlled-release carvedilol capsules are prepared containing controlled-release subunits of Example 5, E5 and H5 prepared from the immediate release subunits of Example 1, F1 using a dry granulation process. The immediate release subunits have an average tablet weight of about 10 mg and are coated with about 3.0 weight percent Opadry Clear prior to the controlled-release coating. The controlled-release subunits of E5 contain about 4.5 weight percent copolymer; and the controlled-release subunits of H5 contain about 10 weight percent copolymer. Subunits of E5 and H5 are combined with talc and loaded into gelatin capsules. Each capsule contains 80 mg carvedilol phosphate with a weight ratio of E5 to H5 subunits of about 3.6:1.

### Example 10

### Bioavailability Study-Fasted Conditions

A single dose, open-label, randomized, 2-period, 2-treatment, 2-way crossover bioavailability study is made with the carvedilol 80 mg capsules of Example 9 under fasted conditions. Fifty-eight healthy subjects are randomly assigned to a treatment sequence and received two separate single-dose administrations of study medication, one treatment per period, according to the randomized schedule. Dosing days are separated by a washout period of at least seven days. The subjects received Treatment A (test formulation of Example 9, 80 mg carvedilol capsule) and Treatment B (reference product, Coreg® CR 80 mg capsule) following an overnight fast of at least ten hours. A blood sample is taken pre-dose, and after administration of the dose, blood samples were taken from the subjects every hour for the first 16 hours, and then at hours 18, 20, 22, 24, 28, 32, 36, 40, 44, and 48. The samples are analyzed for carvedilol and the following pharmacokinetic parameters are calculated: Cₘₐₓ, Tₘₐₓ, AUCₗₐₛₜ the area under the concentration-time curve from time-zero to the time of the last quantifiable concentration, AUC_{inf}, γ_{z} elimination rate constant, and T_{1/2} terminal half-life. The results, provided in Table 8 and Table 9 below, indicate that the formulation of Example 9 exhibits a geometic mean ratio of Cₘₐₓ, AUCₗₐₛₜ and AUC_{inf} of within 80% to 125% limits for carvedilol. The formulation of Example 9 also exhibits a single phase release (single Tₘₐₓ) within the first 10 hours after oral administration.

TABLE 8

| | Treatment A: test formulation | | | Treatment B reference product | | |
|---|---|---|---|---|---|---|
| Parameter | Mean | SD | CV % | Mean | SD | CV % |
| Tₘₐₓ (hr) | 7.71 | 4.07 | 52.73 | 2.64 | 1.99 | 75.38 |
| Cₘₐₓ (ng/ml) | 40.4 | 21.9 | 54.10 | 45.3 | 29.4 | 64.99 |
| AUCₗₐₛₜ (hr*ng/ml) | 574.4 | 278.1 | 48.41 | 537.3 | 307.9 | 57.31 |
| AUC_{inf} (hr*ng/ml) | 631.5 | 310.6 | 49.19 | 595.7 | 343.7 | 57.70 |
| AUC_{Extrap} (%) | 8.17 | 7.08 | 86.75 | 9.26 | 9.43 | 101.78 |
| γ_{z} (hr⁻¹) | 0.0755 | 0.0323 | 42.83 | 0.0719 | 0.0392 | 54.51 |
| T_{1/2} (hr) | 10.84 | 4.51 | 41.60 | 12.57 | 7.60 | 60.51 |
| Tₗₐₛₜ (hr) | 46.69 | 4.13 | 8.85 | 46.49 | 4.84 | 10.42 |
| Cₗₐₛₜ (ng/ml) | 3.02 | 2.56 | 84.80 | 2.61 | 2.36 | 90.43 |

TABLE 9

| | | Geometric | | | | |
|---|---|---|---|---|---|---|
| Dependent | Geometric | mean^{a}: | Ratio (%)^{b} | 90% CI^{c} | | ANOVA |
| Variable | mean^{a}: test | reference | (Test/ref) | (lower, upper) | Power | CV % |
| Ln(Cₘₐₓ) | 35.8095 | 38.9688 | 91.89 | 83.74, 100.84 | 0.9887 | 30.60 |
| Ln(AUCₗₐₛₜ) | 514.0778 | 469.9717 | 109.38 | 102.25, 117.01 | 0.9999 | 21.97 |
| Ln(AUC_{inf}) | 561.5372 | 521.4020 | 107.70 | 100.22, 115.73 | 0.9996 | 23.49 |

^{a}Geometric Mean for the Test Formulation (Test) and Reference Product (Ref) based on Least Square Mean of log-transformed parameter values
^{b}Ratio (%) = Geometric Mean (Test)/Geometric Mean (Ref)
^{c}90% Confidence Interval

### Example 11

### Bioavailability Study-Fed Conditions

A single dose, open-label, randomized, 2-period, 2-treatment, 2-way crossover bioavailability study is made with the carvedilol 80 mg capsules of Example 9 under fed conditions. Sixty-one healthy subjects are randomly assigned to a treatment sequence and received two separate single-dose administrations of study medication, one treatment per period, according to the randomized schedule. Dosing days are separated by a washout period of at least seven days. The subjects received Treatment A (test formulation of Example 9, 80 mg carvedilol capsule) and Treatment B (reference product, Coreg® CR 80 mg capsule) following an overnight fast of at least ten hours and then a standard high-calorie, high-fat breakfast meal taken thirty minutes prior to each dose. A blood sample is taken pre-dose, and after administration of the dose, blood samples were taken from the subjects every hour for the first 16 hours, and then at hours 18, 20, 22, 24, 28, 32, 36, 40, 44, and 48. The samples are analyzed for carvedilol and the following pharmacokinetic parameters are calculated: Cₘₐₓ, Tₘₐₓ, AUCₗₐₛₜ, AUC_{inf}, γ_{z}, and T_{1/2}. The results, provided in Table 10 and Table 11 below, indicate that the formulation of Example 9 exhibits a geometic mean ratio of Cₘₐₓ, AUCₗₐₛₜ and AUC_{inf} of within 80% to 125% limits for carvedilol. The formulation of Example 9 also exhibits a single phase release (single Tₘₐₓ) within the first 10 hours after oral administration.

TABLE 10

| | Treatment A: test formulation | | | Treatment B reference product | | |
|---|---|---|---|---|---|---|
| Parameter | Mean | SD | CV % | Mean | SD | CV % |
| Tₘₐₓ (hr) | 7.36 | 2.83 | 38.46 | 6.13 | 1.96 | 32.00 |
| Cₘₐₓ (ng/ml) | 108 | 60.0 | 55.75 | 96.0 | 47.4 | 49.38 |
| AUCₗₐₛₜ (hr*ng/ml) | 864.2 | 409.5 | 47.39 | 886.6 | 433.9 | 48.93 |
| AUC_{inf} (hr*ng/ml) | 908.4 | 440.2 | 48.45 | 918.5 | 461.0 | 50.19 |
| AUC_{Extrap} (%) | 4.29 | 3.99 | 92.99 | 3.09 | 2.51 | 81.07 |
| γ_{z} (hr⁻¹) | 0.0862 | 0.0317 | 36.76 | 0.0864 | 0.0254 | 29.39 |
| T_{1/2} (hr) | 9.16 | 3.60 | 39.32 | 8.87 | 3.38 | 38.09 |
| Tₗₐₛₜ (hr) | 46.83 | 3.22 | 6.87 | 46.82 | 3.21 | 6.86 |
| Cₗₐₛₜ (ng/ml) | 2.92 | 3.33 | 114.12 | 2.23 | 2.40 | 107.39 |

TABLE 11

| | | Geometric | | | | |
|---|---|---|---|---|---|---|
| Dependent | Geometric | mean^{a}: | Ratio (%)^{b} | 90% CI^{c} | | ANOVA |
| Variable | mean^{a}: test | reference | (Test/ref) | (lower, upper) | Power | CV % |
| Ln(Cₘₐₓ) | 95.0123 | 85.6545 | 110.93 | 101.43, 121.31 | 0.9923 | 30.23 |
| Ln(AUCₗₐₛₜ) | 781.8660 | 796.5198 | 98.16 | 92.84, 103.79 | 1.0000 | 18.58 |
| Ln(AUC_{inf}) | 817.6619 | 822.0768 | 99.46 | 94.24, 104.97 | 1.0000 | 17.96 |

^{a}Geometric Mean for the Test Formulation (Test) and Reference Product (Ref) based on Least Square Mean of log-transformed parameter values
^{b}Ratio(%) = Geometric Mean (Test)/Geometric Mean (Ref)
^{c}90% Confidence Interval

### Example 12

### Bioavailability Study-Sprinkle

A single dose, open-label, randomized, 2-period, 2-treatment, 2-way crossover bioavailability study is made with the carvedilol 80 mg capsules of Example 9 administered orally as a sprinkle on applesauce following an overnight fast of at least ten hours. Sixty healthy subjects are randomly assigned to a treatment sequence and received two separate single-dose administrations of study medication, one treatment per period, according to the randomized schedule. Dosing days are separated by a washout period of at least seven days. The subjects received Treatment A (test formulation of Example 9, 80 mg carvedilol capsule) and Treatment B (reference product, Coreg® CR 80 mg capsule) following an overnight fast of at least ten hours prior to each dose. A blood sample is taken pre-dose, and after administration of the dose, blood samples were taken from the subjects every hour for the first 16 hours, and then at hours 18, 20, 22, 24, 28, 32, 36, 40, 44, and 48. The samples are analyzed for carvedilol and the following pharmacokinetic parameters are calculated: Cₘₐₓ, Tₘₐₓ, AUCₗₐₛₜ, AUC_{inf}, γ_{z}, and T_{1/2}. The results, provided in Table 12 and Table 13 below, indicate that the formulation of Example 9 exhibits a geometic mean ratio of Cₘₐₓ, AUCₗₐₛₜ and AUC_{inf} of within 80% to 125% limits for carvedilol. The formulation of Example 9 also exhibits a single phase release (single Tₘₐₓ) within the first 10 hours after oral administration.

TABLE 12

| | Treatment A: test formulation | | | Treatment B reference product | | |
|---|---|---|---|---|---|---|
| Parameter | Mean | SD | CV % | Mean | SD | CV % |
| Tₘₐₓ (hr) | 6.01 | 2.24 | 37.27 | 3.21 | 1.72 | 53.62 |
| Cₘₐₓ (ng/ml) | 43.4 | 24.2 | 55.70 | 48.6 | 26.0 | 53.38 |
| AUCₗₐₛₜ (hr*ng/ml) | 610.9 | 324.6 | 53.14 | 563.5 | 313.8 | 55.68 |
| AUC_{inf} (hr*ng/ml) | 666.8 | 358.8 | 53.81 | 634.1 | 365.5 | 57.64 |
| AUC_{Extrap} (%) | 8.01 | 7.15 | 89.33 | 9.32 | 10.73 | 115.23 |
| γ_{z} (hr⁻¹) | 0.0766 | 0.0344 | 44.86 | 0.0717 | 0.0388 | 54.07 |
| T_{1/2} (hr) | 11.10 | 5.91 | 53.24 | 13.48 | 9.35 | 69.38 |
| Tₗₐₛₜ (hr) | 47.21 | 3.28 | 6.95 | 45.67 | 5.33 | 11.67 |
| Cₗₐₛₜ (ng/ml) | 3.06 | 2.79 | 91.22 | 2.58 | 2.47 | 95.56 |

TABLE 13

| | | Geometric | | | | |
|---|---|---|---|---|---|---|
| Dependent | Geometric | mean^{a}: | Ratio (%)^{b} | 90% CI^{c} | | ANOVA |
| Variable | mean^{a}: test | reference | (Test/ref) | (lower, upper) | Power | CV % |
| Ln(Cₘₐₓ) | 36.8971 | 41.8496 | 88.17 | 81.73, 95.11 | 0.9990 | 25.21 |
| Ln(AUCₗₐₛₜ) | 530.4812 | 483.4375 | 109.73 | 103.38, 116.47 | 1.0000 | 19.71 |
| Ln(AUC_{inf}) | 578.6862 | 537.2238 | 107.72 | 100.85, 115.06 | 0.9999 | 21.84 |

^{a}Geometric Mean for the Test Formulation (Test) and Reference Product (Ref) based on Least Square Mean of log-transformed parameter values
^{b}Ratio(%) = Geometric Mean (Test)/Geometric Mean (Ref)
^{c}90% Confidence Interval

The terms “a” and “an” do not denote a limitation of quantity, but rather denote the presence of at least one of the referenced item.

The term “or” means “and/or”.

The terms “comprising”, “having”, “including”, and “containing” are to be construed as open-ended terms (i.e., meaning “including, but not limited to”).

The endpoints of all ranges directed to the same component or property are inclusive and independently combinable.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A controlled-release composition, comprising
a plurality of controlled-release subunits comprising a carvedilol phosphate salt or a hydrate solvate, or polymorph form thereof, and a release-retarding coating material, and
a plurality of immediate release subunits comprising a carvedilol phosphate salt or a hydrate, solvate, or polymorph form thereof and an excipient;
wherein the controlled-release composition exhibits a single phase release (single Tₘₐₓ) within the first 10 hours after oral administration to a patient.

2.-6. (canceled)

7. The controlled-release composition of claim 1, wherein the carvedilol phosphate salt is carvedilol dihydrogen phosphate hemihydrate.

8.-13. (canceled)

14. The controlled-release composition of claim 1, wherein the release-retarding coating material is a water insoluble alkyl cellulose; a wax; a shellac; a zein; polyvinyl acetate phthalate; hydroxypropylmethyl-cellulose acetate succinate; cellulose acetate phthalate; methacrylic acid copolymer; hydroxy propyl methylcellulose succinate; cellulose acetate succinate; cellulose acetate hexahydrophthalate; hydroxypropyl methylcellulose hexahydrophthalate; hydroxypropyl methylcellulose phthalate; cellulose propionate phthalate; cellulose acetate maleate; cellulose acetate trimellitate; cellulose acetate butyrate; cellulose acetate propionate; a copolymer of acrylic and methacrylic acid; methacrylic acid-methyl methacrylate copolymer; methacrylic acid-ethyl acrylate copolymer; or a combination comprising at least one of the foregoing materials.

15. The controlled-release composition of claim 1, wherein the release-retarding coating material is ethyl cellulose, a methacrylic acid-methyl methacrylate copolymer, a methacrylic acid-ethyl acrylate copolymer, or a combination comprising at least one of the foregoing materials.

16.-19. (canceled)

20. The controlled-release composition of claim 1, wherein the controlled-release subunits have a mean diameter of greater than about 2100 micrometers.

21. The controlled-release composition of claim 1, wherein the controlled-release subunits have a mean diameter of greater than about 3000 micrometers.

22. (canceled)

23. The controlled-release composition of claim 1, wherein the controlled-release subunits have a mean diameter of about 1000 to about 5000 micrometers.

24. The controlled-release composition of claim 1, wherein the controlled-release subunits have a mean diameter of about 2500 to about 5000 micrometers.

25. (canceled)

26. The controlled-release composition of claim 1, wherein the controlled-release subunits and the immediate release subunits are prepared by compression.

27.-30. (canceled)

31. The controlled-release composition of claim 1, wherein the composition is a capsule comprising immediate-release subunits and controlled-release subunits.

32. The controlled-release composition of claim 1, wherein the composition is a tablet comprising immediate-release subunits and controlled-release subunits.

33. The controlled-release composition of claim 1, wherein the carvedilol is carvedilol dihydrogen phosphate hemihydrate present in an amount of about 60 to about 100 mg free base equivalent based on the total weight of the composition.

34.-41. (canceled)

42. The controlled-release composition of claim 1, wherein the composition provides one or more of the following:
a mean plasma level of carvedilol of about 7 ng/ml or more 1 hour after oral administration to a patient;
a mean plasma level of carvedilol of about 7 ng/ml or greater for a 24 hour period after oral administration to a patient;
a geometric mean of logarithmic transformed AUC_{0-∞} of about 500 ng.h/ml to about 600 ng.h/ml;
a geometric mean of logarithmic transformed AUC_{0-∞} of the controlled-release composition that is within about 80% and about 125% of 525 ng·b/ml; or
a geometric mean of logarithmic transformed Cₘₐₓ of the controlled-release composition that is within about 80% and about 125% of 54 ng/ml.

43.-53. (canceled)

54. The controlled-release composition of claim 1, wherein the composition is bioequivalent to a reference drug according to New Drug Application No. 022012.

55. A method of treating hypertension, congestive heart failure, or angina by administering a controlled-release composition of carvedilol according to claim 1.

56. The controlled-release composition of claim 1, wherein a dissolution profile of the composition is substantially the same as a dissolution profile of an equivalent strength of a reference drug according to New Drug Application No. 022012.

57. The controlled-release composition of claim 56, wherein the dissolution profile is determined using the conditions according to USP 28 <711> test method 2 (paddle) using of 900 ml of a dissolution medium at 37° C.±2° C., and 50 rpm paddle speed, and wherein the dissolution medium is purified water, 0.1 N HCl, 4.5 pH buffer, or 6.8 pH buffer.

58.-65. (canceled)

66. A controlled-release capsule composition, comprising:
a plurality of controlled-release subunits prepared by compression comprising a carvedilol phosphate salt or a hydrate, solvate, or polymorph form thereof, and a methacrylic acid-methyl methacrylate copolymer as a release-retarding coating material,
a plurality of controlled-release subunits prepared by compression comprising a carvedilol phosphate salt or a hydrate, solvate, or polymorph form thereof, and a methacrylic acid-ethyl acrylate copolymer as a release-retarding coating material; and
a plurality of immediate release subunits prepared by compression comprising a carvedilol phosphate salt or a hydrate, solvate, or polymorph form thereof and an excipient;
wherein the controlled-release subunits have a mean diameter of greater than about 2100 micrometers,
wherein the capsule comprises about 60 to about 100 mg free base equivalent of carvedilol, and
wherein the controlled-release composition exhibits a single phase release (single Tₘₐₓ) within the first 10 hours after oral administration to a patient.

67. The controlled-release composition of claim 66, wherein the carvedilol phosphate salt is carvedilol dihydrogen phosphate hemihydrate.

68. The controlled-release composition of claim 66, wherein the capsule comprises about 80 mg free base equivalent of carvedilol.
